(19) United States
(12) Patent Application Publication
(10) Pub. No.: US 2010/0185001 A1
(43) Pub. Date: Jul. 22, 2010

(54) PROCESS AND APPARATUS FOR THE PRODUCTION OF ETHYLENE OXIDE

(76) Inventors: Wouter VAN MAAREN, Amsterdam (NL); Franciscus Johannes Maria Schrauwen, Amsterdam (NL)

Correspondence Address: SHELL OIL COMPANY, P O BOX 2463, HOUSTON, TX, 772522463, US

(21) Appl. No.: 12/689,143

(22) Filed: Jan. 18, 2010

(30) Foreign Application Priority Data
Jan. 19, 2009 (EP) 09150864.8

(51) Int. Cl. C07D 301/03; B01J 19/24

(52) U.S. Cl. 549/523; 422/198

(57) ABSTRACT

The invention provides a process and an apparatus for the production of ethylene oxide from ethylene. Ethylene and oxygen are supplied to reactor tubes, wherein the reactor tubes are held by upper and lower tube sheets in a reactor vessel. The reactor vessel has a separation grid, dividing the reactor vessel into an upstream zone and a downstream zone. Coolant is supplied to the upstream zone from an upper coolant circuit and is removed from the upstream zone to the upper coolant circuit. A portion of coolant is removed as vapour from the upper coolant circuit. Coolant is supplied to the downstream zone from a lower coolant circuit and is removed from the downstream zone to the lower coolant circuit. Additional coolant is added to the lower coolant circuit. There is net flow of coolant through the separation grid from the downstream zone to the upstream zone.

This application claims the benefit of European Application No. 09150864.8 filed Jan. 19, 2009 which is incorporated herein by reference.

### FIELD OF THE INVENTION

The present invention relates to a process and apparatus for the production of ethylene oxide.

### BACKGROUND OF THE INVENTION

Ethylene oxide is used as a chemical intermediate, primarily for the production of ethylene glycols but also for the production of ethoxylates, ethanol-amines, solvents and glycol ethers. It is produced by the direct oxidation of ethylene with oxygen or air. Ethylene and oxygen are passed over a silver oxide catalyst, typically at pressures of 10-30 bar and temperatures of 200-300° C. The reaction is exothermic and a typical reactor consists of an assembly of tubes that are packed with catalyst. A coolant surrounds the reactor tubes, removing the reaction heat and permitting temperature control.

Reactor designs have been developed to reduce the formation of by-products, thereby improving the quality of the ethylene oxide product. In U.S. Pat. No. 3,147,084, the reactor is transversely divided by a tube sheet into an upstream reaction zone and a downstream cooling zone. Heat-exchange fluids are separately circulated in the two isolated zones, although a small seepage of fluid between the zones is permissible. In U.S. Pat. No. 5,292,904, the reactor is transversely divided by a partition plate into a reaction zone and a cooling zone. Hot water, used as the heat exchange fluid in the cooling zone, is supplied to a gas-liquid separation tank, and hot water from the tank is supplied to the reaction zone.

EP 821 678 discloses a system using a single-compartment reactor wherein some or all of the heat exchange fluid is introduced into the reactor at its downstream end at a temperature which is at least 20° C. below the temperature of the heat exchange fluid on leaving the reactor. EP 1 358 441 discloses a tubular reactor of conventional type in conjunction with a heat exchanger which is integral with the discharge head of the tubular reactor.

U.S. Pat. No. 4,203,906 discloses a reactor wherein a perforated shield plate divides the reactor into two heat transfer zones. The heat exchange fluid can flow through the perforated shield plate between the heat transfer zones, yet it is possible to retain a temperature difference between the two zones. There is no substantial movement of heat transfer medium between the two zones. There is no substantial movement of heat transfer medium between the two zones.

EP 0 911 313 discloses a reactor wherein a partition plate divides the reactor into an upper space and lower space, allowing substantially independent circulation of a heating medium in the upper space and lower space. This provides temperature control of catalyst layers in reaction tubes.

EP 0 911 313 discloses a reactor wherein a partition plate divides the reactor into an upper space and lower space, allowing substantially independent circulation of a heating medium in the upper space and lower space. This provides temperature control of catalyst layers in reaction tubes.

The present inventors have sought to provide an apparatus and a process for the production of ethylene oxide from ethylene, wherein the amount of by-products is minimized. Desirably the apparatus has a simple and cost-effective design and enables the operation of an energy efficient process.

### SUMMARY OF THE INVENTION

Accordingly, the present invention provides apparatus for the production of ethylene oxide from ethylene, comprising

a reactor vessel having reactor tubes held by upper and lower tube sheets;

a separation grid, dividing the reactor vessel into an upstream zone and a downstream zone;

an upper coolant circuit, whereby coolant can be supplied to the upstream zone, removed from the upstream zone and recirculated to the upstream zone, and wherein a portion of coolant can be removed as vapour from the upper coolant circuit; and

a lower coolant circuit, whereby coolant can be supplied to the downstream zone, removed from the downstream zone, and recirculated to the downstream zone, and wherein additional coolant can be added to the lower coolant circuit;

wherein the separation grid allows the net flow of coolant from the downstream zone to the upstream zone.

The invention further provides a process for the production of ethylene oxide from ethylene comprising steps of:

a) supplying ethylene and oxygen to reactor tubes, wherein the reactor tubes are held by upper and lower tube sheets in a reactor vessel, and wherein the reactor vessel has a separation grid, dividing the reactor vessel into an upstream zone and a downstream zone;
b) supplying coolant to the upstream zone from an upper coolant circuit, removing coolant from the upstream zone to the upper coolant circuit, and removing a portion of coolant as vapour from the upper coolant circuit; and
c) supplying coolant to the downstream zone from a lower coolant circuit, removing coolant from the downstream zone to the lower coolant circuit and adding additional coolant to the lower coolant circuit;
wherein there is net flow of coolant through the separation grid from the downstream zone to the upstream zone.

In prior art systems wherein heat-exchange fluids are separately circulated in two isolated zones separated by a partition tube sheet, it is possible to retain a significant temperature differential between the two zones. This has the advantage that the downstream zone can be significantly cooler than the upstream zone so that the formation of by-products can be reduced and the quality of the ethylene oxide product can be improved. However, reactors with partition tube sheets are expensive and there are mechanical complications associated with the use and construction of the partition tube sheets. The inventors have found that in the apparatus and process of the invention it is possible to retain a significant temperature differential between the upstream and downstream zones, even though the reactor contains a simple separation grid instead of a fixed tube sheet. By controlling the coolant circuits such that there is net flow of coolant through the separation grid from the downstream zone to the upstream zone, there is little or no flow of coolant from the hotter upstream zone to the cooler downstream zone, and the temperature differential can be maintained. Therefore, the apparatus and process of the invention can be used to provide ethylene oxide of high quality with few byproducts.

### BRIEF DESCRIPTION OF THE DRAWINGS

FIG. 1 is a schematic diagram showing an apparatus according to the invention.

### DETAILED DESCRIPTION OF THE INVENTION

Ethylene and oxygen are supplied to a reactor vessel having reactor tubes held by upper and lower tube sheets. The oxygen may be supplied as oxygen or as air, but is preferably supplied as oxygen. A ballast gas, for example methane, is preferably also supplied to allow operation at high oxygen levels without causing a flammable mixture. A moderator, e.g. monochloroethane, vinylchloride or dichloroethane, may be supplied for catalyst performance control. The ethylene, oxygen, ballast gas and moderator are preferably supplied to recycle gas that is supplied to the reactor vessel from an ethylene oxide absorber.

The reactor vessel preferably contains from 1000 to 20000 reactor tubes, preferably from 2500 to 15000 reactor tubes. The reactor tubes preferably have a length in the range of from 5 to 20m, more preferably from 10 to 15m, and preferably have an internal diameter in the range of from 15 to 80 mm, more preferably from 20 to 75 mm, and most preferably from 25 to 70 mm. The reactor tubes are preferably substantially vertical such that they are preferably no more than 5° from vertical.

The upper ends of the reactor tubes are connected to an upper tube sheet and are in fluid communication with the one or more inlets to the reactor vessel and the lower ends of the reactor tubes are connected to a lower tube sheet and are in fluid communication with the one or more outlets to the reactor vessel. The upper and lower tube sheets are preferably substantially horizontal such that they are preferably no more than 3° from horizontal.

The reactor tubes contain a catalyst bed. Particles which may be contained in the catalyst bed other than the catalyst particles are, for example, inert particles. The catalyst bed is preferably supported in the reactor tubes by a catalyst support means arranged in the lower ends of the reactor tubes. The support means may include a screen or a spring.

The reactor tubes optionally also contain one or more separate beds of particles of an inert material for the purpose of heating a feedstream or for cooling of the reaction product. Alternatively, rod-shaped metal inserts may be used in place of a bed of inert material. For further description of such inserts, reference is made to U.S. Pat. No. 7,132,555.

The preferred catalyst particles comprise silver deposited on a carrier. Suitable carrier materials include refractory materials, such as alumina, magnesia, zirconia, silica, and mixtures thereof. The catalyst particles may also comprise a promoter component, e.g. rhenium, tungsten, molybdenum, chromium, nitrate- or nitrite-forming compounds, and combinations thereof.

The separation grid divides the reactor vessel into an upstream zone and a downstream zone and allows the net flow of coolant from the downstream zone to the upstream zone. The separation grid is a plate having holes, through which the reaction tubes can pass. The reaction tubes are not connected (e.g. welded) to the separation grid, although there can be contact between the reaction tubes and the separation grid. Thus, one part of a reactor tube can be in the upstream zone and another part of the same reactor tube can be in the downstream zone.

When the separation grid is installed in the reactor vessel such that the reaction tubes pass through the holes in the separation grid, it has an open area such that coolant can pass through the separation grid. The holes in the separation grid are larger than the external diameter of the reactor tubes such that the reactor tubes pass easily through the holes (a typical tolerance is from 0.2 to 3 mm), and the gap between the tubes and the separation grid provides some or all of the open area of the separation grid. In addition, there may be additional holes in the separation grid (through which reactor tubes do not pass) that also provide some of the open area of the separation grid. Preferably the open area of the separation grid represents from 0.5 to 8% of the cross section of the reactor vessel, more preferably from 1 to 5% and most preferably from 1 to 3%. A smaller open area would be difficult to achieve because of the manufacturing tolerance associated with making the holes in the separation grid such that the reactor tubes will pass through during assembly, and a larger open area is not preferred because this may allow for significant flow of coolant from the upstream zone to the downstream zone, which may lead to heating of the downstream zone and increased byproduct formation.

The separation grid is preferably metal and is more preferably made from a single sheet of metal. The most preferred metal is carbon steel. The thickness of the separation grid is preferably less than 100 mm, more preferably from 5 to 50 mm and most preferably from 10 to 30 mm.

The separation grid is preferably suspended from the upper tube sheet by vertical rods. Preferably, these rods also carry conventional tube support grids that are in place to assure position of the tubes, typically every 1.5-2.5m of height. Conventional tube support grids are plates having holes, through which the reaction tubes can pass, and additionally having holes for the passage of steam and water. The difference between the conventional tube support grids and the separation grid, is that the conventional tube support grids are designed to allow significant flow of fluids through the grid whereas the separation grid is designed to allow only a limited flow of fluids through the grid. The open area of the tube support grids typically represents from 20 to 30% of the cross section of the reactor vessel.

Preferably, there is a small gap, e.g. 1-5 mm between the circumference of the separation grid and the shell of the reactor vessel. This is to enable installation and to accommodate differential thermal expansion.

The separation grid divides the reactor vessel into an upstream zone and a downstream zone. The preferred vertical position of the separation depends upon the efficiency of cooling, flow conditions and temperature of the coolant in the downstream zone. However, the separation grid preferably divides the reactor vessel into an upstream zone representing from 50 to 95% of the reactor vessel volume, and a downstream zone representing 5 to 50% of the reactor vessel volume. More preferably the upstream zone represents from 70 to 90% of the reactor vessel volume and the downstream zone represents from 10 to 30% of the reactor vessel volume.

In a preferred embodiment of the invention, the reactor tubes comprise a catalyst bed that is positioned wholly within the upstream zone and the reactor tubes contain inert materials such as inert particles or reactor inserts within the downstream zone. The inert materials in the downstream zone enhance heat transfer from the reactor product gas to the coolant, thereby reducing byproduct formation in the downstream zone. The reactor tubes preferably also comprise a bed of inert material in the upstream zone, which is upstream of the catalyst bed. Such an arrangement enhances transfer of heat from the coolant in the upstream zone to the feed gases.

Thus, in one embodiment the reactor tubes can be substantially free of catalyst in the downstream zone. By “substantially free of catalyst” it is meant that the part of a reactor tube in the downstream zone does not contain a, or a part of a, catalyst bed, but may contain minor levels of catalyst e.g. catalyst entrained in the gas stream from a catalyst bed positioned in the upstream zone and transferred to the downstream zone. In a further embodiment, the reactor tubes can be wholly free of catalyst in the downstream zone.

An upper coolant circuit supplies coolant to and removes coolant from the upstream zone. In the upper coolant circuit a portion of coolant is removed as vapour before the coolant is recirculated to the upstream zone. A lower coolant circuit supplies coolant to and removes coolant from the downstream zone. In the lower coolant circuit, additional coolant is added before the coolant is recirculated to the downstream zone. By adding additional coolant to downstream zone, by removing coolant as vapour from the upstream zone and by having a separation grid having an open area that permits passage of coolant through the separation grid, it is possible to ensure net flow of coolant from the downstream zone to the upstream zone.

The coolant in the upper and lower coolant circuits is essentially the same material as there is passage of coolant from the downstream zone to the upstream zone. The coolant is a coolant that can be removed as a vapour. The coolant can be a hydrocarbon or a mixture of hydrocarbons such as n-octane, n-nonane, kerosene, ISOPAR™, MOBILTHERM™ or DOWTHERM™, but preferably the coolant is an aqueous material, most preferably water.

Coolant is supplied to the lower coolant circuit, preferably as a liquid, and is removed from the upper coolant circuit as a vapour. Preferably the rates of supply and removal are the same or similar such that the amount of coolant in the system is approximately constant. The rate of supply of coolant is determined at least partly by the amount of cooling that is required. Much of the process heat that is generated is removed by the coolant, although there may also be a temperature difference between the products and the reactants, such that process heat is also removed by the products.

In one embodiment of the invention, additional coolant is supplied to the upper coolant circuit, preferably as a liquid. In this embodiment, the combined rate of supply of coolant to the upper and lower coolant circuits is preferably the same or similar to the rate of removal of coolant from the upper coolant circuit. Even though coolant is supplied to the upper coolant circuit, it is still possible to ensure net flow of coolant from the downstream zone to the upstream zone by removing coolant from the upper coolant circuit. Adding coolant at two positions (into the lower and upper coolant circuits) provides additional flexibility in controlling the flow of coolant through the separation grid.

The reaction of ethylene with oxygen to provide ethylene oxide is exothermic. Additionally, there are strongly exothermic side reactions, such as the combustion of ethylene and ethylene oxide to carbon dioxide and water. In the present invention, most reaction will occur in the upstream zone of the reactor vessel and therefore it is necessary to remove the heat of reaction in the upstream zone to ensure that the reaction proceeds at the desired temperature with the desired selectivity. Furthermore, rapid cooling of the products of the oxidation reaction ensures that production of by-products such as aldehydes is minimized, and therefore it is necessary to cool the downstream zone of the reactor vessel. It is desirable to maintain a temperature differential between the coolant in the downstream zone and the coolant in the upstream zone. The temperature of the coolant in the downstream zone is preferably at least 5° C. less than the temperature of the coolant in the upstream zone, more preferably at least 10° C. less, yet more preferably at least 20° C. less and most preferably at least 30° C. less. The temperature in the downstream zone is preferably between 150° C. and 250° C., more preferably between 160° C. and 230° C., and most preferably between 170° C. and 210° C. The temperature in the upstream zone is preferably between 180° C. and 325° C., more preferably between 200° C. and 300° C. and most preferably between 220° C. and 300° C.

In the upper coolant circuit, a portion of coolant is removed as vapour. In the preferred embodiment of the invention wherein the coolant is water, the portion of coolant is removed as steam. Cooling in the upstream zone is at least partly achieved by evaporation of the coolant in the upstream zone such that some of the coolant that is removed from the upstream zone is in the form of vapour and some is in the form of liquid. A portion of the coolant is removed from the upper coolant circuit as vapour. Most preferably there is a steam drum in the upper coolant circuit. Steam is separated from the water/steam mixture entering the steam drum, and steam is withdrawn from the steam drum. The water is recirculated to the upstream zone. Preferably the coolant is introduced from the upper coolant circuit to the upstream zone via one or more coolant injection spargers or nozzles along the circumference of the reactor. Preferably the coolant is introduced to the upstream zone at the bottom of the upstream zone (near the separation grid) and is removed from the upstream zone near the top of the reactor vessel.

In the embodiment of the invention wherein additional coolant is supplied to the upper coolant circuit, it is preferred that additional water is supplied to a steam drum. The steam drum preferably has a liquid level control, such that additional water is added if the liquid falls below a set level.

In the lower coolant circuit, coolant is supplied at a temperature below the temperature of the coolant in the upstream zone. Continuous supply of coolant at a lower temperature allows the removal of the process heat. In addition to the supply of coolant, heat removal is preferably achieved by subjecting the coolant in the lower coolant circuit to heat exchange. The heat exchange is most preferably conducted by a trim cooler in the lower coolant circuit. When a trim cooler is used, it is desirable that the temperature of the coolant leaving the downstream zone is sufficiently high that the trim cooler can be used to generate steam or can be used to heat a process stream. Preferably the coolant in the downstream zone does not evaporate, and the coolant in the downstream zone and in the lower coolant circuit is present as liquid. There is preferably also a coolant circulation pump in the lower coolant circuit to recirculate the coolant. Preferably the coolant is introduced from the lower coolant circuit to the downstream zone via one or more coolant injection spargers or nozzles along the circumference of the reactor. Preferably the coolant is introduced to the downstream zone near the bottom of the reactor vessel and is removed from the downstream zone near the top of the downstream zone (near the separation grid). For small systems, it is likely to be sufficient to introduce coolant at the perimeter of the reactor vessel, but for larger systems it may be preferable to use radial distribution tubes as this can provide improved distribution of coolant across all of the reactor tubes.

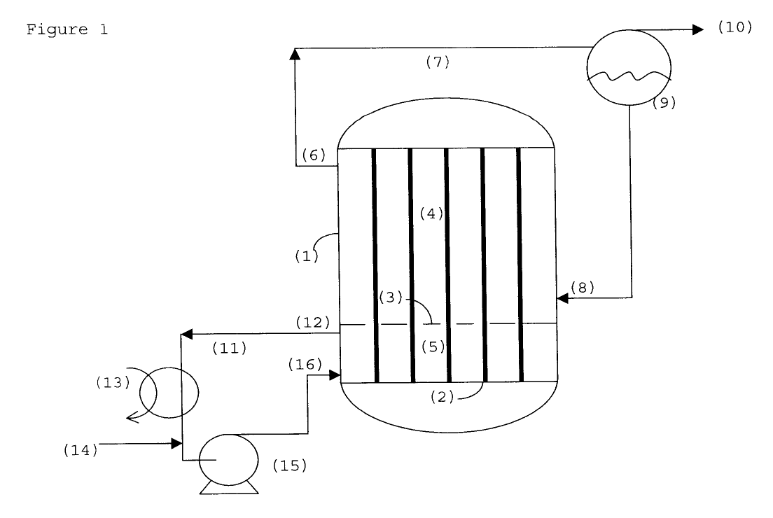

FIG. 1 shows a preferred embodiment of the apparatus the invention. Reactor tubes (2) are contained within a reactor vessel (1), and are connected to upper and lower tube sheets. A separation grid (3) is positioned within the reactor vessel (1), dividing the reactor vessel (1) into an upstream zone (4) and a downstream zone (5).

An upper coolant circuit (7) contains a steam drum (9). A lower coolant circuit (11) contains a trim cooler (13) and a coolant circulation pump (15).

Ethylene and oxygen are supplied to the top of the reactor tubes (2) in the reactor vessel (1). The ethylene and oxygen react in an exothermic reaction to provide ethylene oxide. This reaction occurs predominantly in the tubes in the upstream zone (4) and significant heat is produced in the upstream zone (4). The reaction products pass through the reactor tubes (2) in the downstream zone (5) and it is desirable to rapidly cool products passing through the downstream zone (5) to reduce by-product formation.

Water is supplied (14) to the lower coolant circuit (11). Water is pumped through the lower coolant circuit (11) by the coolant circulation pump (15) and is supplied (16) to the downstream zone (5). The water will take up heat by contact with the reactor tubes (2) in the downstream zone, so will be hotter when withdrawn (12) from the downstream zone (5) than when it is supplied (16) to the downstream zone (5). In the lower coolant circuit the trim cooler (13) removes further heat from the water before it is re-supplied to the downstream zone (5).

Water in the upstream zone (4) is heated by contact with the reactor tubes (2). The heat of reaction is such that water in the upstream zone (4) will evaporate. A mixture of steam and liquid water is withdrawn (7) from the upstream zone (4) to the upper coolant circuit (6) and supplied to the steam drum (9). Steam is withdrawn (10) from the steam drum (9). The water is recirculated and supplied (8) to the upstream zone (4).

Water is supplied (14) to the lower coolant circuit (11) and steam is withdrawn (10) from the steam drum (9) in the upper coolant circuit (6). Because of this, there is net flow of water through the separation grid (3), from the downstream zone (5) to the upstream zone (4). There is little or no flow of coolant from the hotter upstream zone (4) to the cooler downstream zone (5), and the temperature differential between the zones is maintained.

What is claimed is:

1. An apparatus for the production of ethylene oxide from ethylene, comprising
a reactor vessel having reactor tubes held by upper and lower tube sheets;
a separation grid, dividing the reactor vessel into an upstream zone and a downstream zone;
an upper coolant circuit, whereby coolant can be supplied to the upstream zone, removed from the upstream zone and recirculated to the upstream zone, and wherein a portion of coolant can be removed as vapour from the upper coolant circuit; and
a lower coolant circuit, whereby coolant can be supplied to the downstream zone, removed from the downstream zone and recirculated to the downstream zone, and wherein additional coolant can be added to the lower coolant circuit;
wherein the separation grid allows the net flow of coolant from the downstream zone to the upstream zone.

2. An apparatus according to claim 1 wherein the open area of the separation grid represents from 0.5 to 8% of the cross section of the reactor vessel.

3. An apparatus according to claim 1 wherein the upstream zone represents from 50 to 95% of the reactor vessel volume, and the downstream zone represents from 5 to 50% of the reactor vessel volume.

4. An apparatus according to claim 1 wherein there is a steam drum in the upper coolant circuit.

5. An apparatus according to claim 1 wherein the coolant in the lower coolant circuit is subjected to heat exchange.

6. An apparatus according to claim 5 wherein there is a trim cooler and a pump in the lower coolant circuit.

7. An apparatus according to claim 1 wherein the reactor tubes comprise a catalyst bed that is positioned wholly within the upstream zone.

8. An apparatus according to claim 7 wherein the reactor tubes are substantially free of catalyst in the downstream zone.

9. A process for the production of ethylene oxide from ethylene comprising steps of:
a) supplying ethylene and oxygen to reactor tubes, wherein the reactor tubes are held by upper and lower tube sheets in a reactor vessel, and wherein the reactor vessel has a separation grid, dividing the reactor vessel into an upstream zone and a downstream zone;
b) supplying coolant to the upstream zone from an upper coolant circuit, removing coolant from the upstream zone to the upper coolant circuit, and removing a portion of coolant as vapour from the upper coolant circuit; and
c) supplying coolant to the downstream zone from a lower coolant circuit, removing coolant from the downstream zone to the lower coolant circuit and adding additional coolant to the lower coolant circuit;
wherein there is net flow of coolant through the separation grid from the downstream zone to the upstream zone.

10. A process according to claim 9 wherein the open area of the separation grid represents from 0.5 to 8% of the cross section of the reactor vessel.

11. A process according to claim 9 wherein the upstream zone represents from 50 to 95% of the reactor vessel volume, and the downstream zone represents from 5 to 50% of the reactor vessel volume.

12. A process according to claim 9 wherein there is a steam drum in the upper coolant circuit.

13. A process according to claim 9 wherein the coolant in the lower coolant circuit is subjected to heat exchange.

14. A process according to claim 13 wherein there is a trim cooler and a pump in the lower coolant circuit.

15. A process according to claim 9 wherein the reactor tubes comprise a catalyst bed that is positioned wholly within the upstream zone.

16. A process according to claim 9 wherein the reactor tubes are substantially free of catalyst in the downstream zone.
